(19) United States
(12) Patent Application Publication
(10) Pub. No.: US 2025/0201405 A1
(43) Pub. Date: Jun. 19, 2025

(54) METHOD FOR PREDICTING PROGNOSTIC INDICATIONS OF PANCREATIC CANCER PATIENTS

(71) Applicant: National Cheng Kung University, Tainan City (TW)

(72) Inventors: Po See Chen, Tainan City (TW); Yan-Shen Shan, Tainan City (TW); Chia-Jui Yen, Tainan City (TW); Ping-Yen Liu, Tainan City (TW); Cheng-Feng Lin, Tainan City (TW)

(21) Appl. No.: 18/810,801

(22) Filed: Aug. 21, 2024

Related U.S. Application Data
(60) Provisional application No. 63/609,684, filed on Dec. 13, 2023.

(51) Int. Cl. G16H 50/20; C12N 15/10; C12Q 1/6806; C12Q 1/686; G01N 33/68; G16B 20/20; G16H 10/20; G16H 10/60

(52) U.S. Cl. CPC G16H 50/20; C12N 15/1096; C12Q 1/6806; C12Q 1/686; G01N 33/6893; G16B 20/20; G16H 10/20; G16H 10/60; C12Q 2600/118; G01N 2333/4737; G01N 2333/705; G01N 2333/765

(57) ABSTRACT

The present invention relates to a method for predicting a prognostic indications of pancreatic cancer patient, comprising: providing a sample, wherein the sample is peripheral blood; isolating the sample to obtain an object, wherein the object includes gene loci, protein or blood cell; detecting a reference parameter from the object for estimating a prognostic indicators of pancreatic cancer patient; and estimating the prognostic indications of pancreatic cancer patient by using a model.

### CROSS-REFERENCE TO RELATED APPLICATIONS

This application claims the benefit of U.S. 63/609,684, filed Dec. 13, 2023, which is incorporated in its entirety by reference herein.

### FIELD OF THE INVENTION

The present invention relates to predict a prognostic indications of a pancreatic cancer patients, particularly relates to a method for predicting a prognostic indications of a pancreatic cancer patients that uses single nucleotide polymorphism of gene loci, expression level of inflammatory marker, and/or clinical manifestations.

### BACKGROUND OF THE INVENTION

Pancreatic cancer is a highly malignant tumor with poor prognosis. Despite significant advancements in modern medical technology and the improved prognosis of many other cancers, pancreatic cancer remains a devastating disease with a prognosis that has remained largely unchanged for the past 20 years. Due to its insidious early symptoms and lack of specific diagnostic methods, most patients are diagnosed over the best time for surgery. Even with surgical treatment, the 5-year survival remains extremely low, and it still lacks effective treatments and methods to predict disease progression.

It is paramount importance for clinical treatment and health economics that timely estimation of treatment effect, disease progression, and prognosis in pancreatic cancer. Currently, most prognosis estimation for pancreatic cancer relies on single indications. However, these indications cannot accurately understand progression and prognosis of the disease and let predict the prognosis in advance, on the contrary it brings difficulties and risks to clinical diagnosis and treatment.

Therefore, it is necessary to develop a reliable and accurate pancreatic cancer prognosis estimation to aim treatment guidance and prognosis prediction, and to improve patients' survival and quality of life. This has become an extremely important issue in the present day.

### SUMMARY OF THE INVENTION

According to the drawbacks of the prior art, the present invention aims to estimate the prevalence of survival among patients with pancreatic cancer.

According to abovementioned, the present invention provides a method for predicting a prognostic indications of pancreatic cancer patient, which includes: providing a sample, wherein the sample is peripheral blood; isolating the sample to obtain an object, wherein the object is a gene loci; detecting a reference parameter from the object for estimating a prognostic indicators of pancreatic cancer patient, wherein the reference parameter is a single nucleotide polymorphism of gene loci; and combining the reference parameter and a clinical manifestation to estimate the prognostic indications of pancreatic cancer patient by using a model.

According to the above objects, the present invention further discloses a method for predicting a prognostic indications of pancreatic cancer patient, which includes: providing a sample, wherein the sample is peripheral blood; isolating the sample to obtain an object, wherein the object is protein or blood cell; detecting a reference parameter from the object for estimating a prognostic indicators of pancreatic cancer patient, wherein the reference parameter is an object expression level of an inflammatory marker from the object to obtain at least one desired protein expression level or a number of blood cells; and combining the reference parameter and a clinical manifestation to estimate the prognostic indications of pancreatic cancer patient by using a model.

Furthermore, the present invention further discloses a method for predicting a prognostic indications of pancreatic cancer patient, which includes: providing a sample, wherein the sample is peripheral blood; isolating the sample to obtain an object, wherein the object includes a gene loci, a protein or a blood cell; detecting a reference parameter from the object for estimating a prognostic indicators of pancreatic cancer patient; and estimating the prognostic indications of pancreatic cancer patient by using a model; wherein the reference parameter comprises at least one of a single nucleotide polymorphism of gene loci, an object expression level of an inflammatory marker, and a clinical manifestation; wherein the single nucleotide polymorphism of gene loci is detected from the object to confirm the single nucleotide polymorphism of a desired gene loci; wherein the object expression level of the inflammatory marker is detected from the object to obtain at least one desired protein expression level or a number of blood cells.

### BRIEF DESCRIPTION OF THE DRAWINGS

FIG. 1 is a schematically flowing diagram illustrating method for predicting a prognostic indications of a pancreatic cancer patients extract according to one embodiment of the present invention.

FIG. 2 is a schematically flowing diagram illustrating method for predicting a prognostic indications of a pancreatic cancer patients extract according to one embodiment of the present invention.

FIG. 3 is a schematically flowing diagram illustrating method for predicting a prognostic indications of a pancreatic cancer patients extract according to one embodiment of the present invention.

### DETAILED DESCRIPTION OF THE PREFERRED EMBODIMENT

The 9-item self-report Patient Health Questionnaire (PHQ-9) assessed the severity of depressive symptoms in the past 2 weeks based on the Diagnostic and Statistical Manual of Mental Disorders-IV (DSM-IV) depression criteria. Responses are on a 4-point scale ranging from 0 (not at all) to 3 (almost every day), giving a total score of 0-27 (PHQ_TP). Regarding depression severity, a total score of 0-4 indicates minimal depression, 5-9 mild depression, 10-14 moderate depression, 15-19 moderately severe depression, and 20-27 severe depression. A depression cutoff of >10 (PHQ Pass) was used in the invention since it is more commonly used among various populations and found to have a sensitivity of 0.77 (0.71 to 0.84) and a specificity of 0.94 (0.90 to 0.97) in screening for major depression in cancer outpatients.

The Montreal Cognitive Assessment (MoCA), a brief screening tool for cognitive impairment, was used to observe cognitive function in the present invention. The MoCA screening tool is a widely used cognitive screening tool designed to assess various cognitive functions, including memory recall, attention, language, and visuospatial abilities. The MoCA assesses six domains of cognitive function including visuospatial/Executive function, naming, attention, language, abstraction, working memory, and orientation. The total score of MoCA (MoCA_TP) is 30, and a cut point of 26 (MoCA_Pass) indicates cognitive impairment, in which when the total score of MoCA_TP is lower than the cut point, the score of MoCA_TP indicates cognitive impairment; when the total score of MoCA_TP is higher than cut point, the score of MoCA_TP indicates no cognitive impairment. In addition, it should be explained to that MoCA_Pass represents the total score is higher than 26 in the table(s) of this invention.

Genetic risk score (GRS) is a number that summarizes the estimated effect of many genetic variants on an individual's phenotype. The score reflects an individual's estimated genetic predisposition for a given trait and can be used as a predictor for that trait. It gives an estimate of how likely an individual is to have a given trait based only on genetics, without taking environmental factors into account; and it is typically calculated as a weighted sum of trait-associated alleles. The way to calculate Genetic risk scores are using the following equation, GRS=Σᵢ₌₁ⁿβᵢ×Gᵢ, βᵢ means weighted coefficient, Gᵢ means risk allele count.

The main purpose of Least Absolute Shrinkage and Selection Operator (LASSO) Regularized Regression is to minimize the sum of the Sum of the Squared Errors (SSE) and the L1 Penalty λΣⱼ₌₁^{p}|βⱼ|, which the L1 Penalty is used to limit the regression coefficients, and turning the coefficients of irrelevant variables into 0, and then automatically conducting the selection of variable. The gene loci are classified as with or without variation. When the zygosity status is NA, it is recorded as 0 (without variation). When the zygosity status is heterozygote (Mm) or homozygotes (MM), it is recorded as 1 (without variation). Due to the uneven proportion of variations in part of gene loci, variations with one class proportion less than 10% will be excluded. Ultimately, a total of 84 gene loci will conduct important gene locus selection by LASSO.

Kaplan-Meier Curve is often used to measure the fraction of patients living for a certain amount of time after treatment. the survival function S(t) (the probability that life is longer than t) is given by:

$$\hat{S} ( t ) = {\prod }_{{εt}_{i} \leq t} ( 1 - \frac{{d}_{i}}{{n}_{i}} ) ,$$

with ti a time when at least one event happened, di the number of events (e.g., deaths) that happened at time tᵢ, and nᵢ the individuals known to have survived (have not yet had an event or been censored) up to time tᵢ.

Cox Proportional Hazards Model is essentially a regression model used statistical in medical research for investigating the association between the survival time of patients and predictor variables. The purpose of Cox Proportional Hazards Model is to evaluate simultaneously the effect of several factors on survival. In other words, it allows us to examine how specified factors influence the rate of a particular event happening (e.g., infection, death) at a particular point in time. This rate is commonly referred as the hazard rate. The Cox Proportional Hazards Model is expressed by the hazard function denoted by h(t). Briefly, the hazard function can be interpreted as the risk of dying at time t. It can be estimated as follow equation: h(t)=h₀(t)>exp(b₁x₁+b₂x₂+ . . . +bₚxₚ). t represents the survival time. h(t) is the hazard function determined by a set of p covariates (x₁, x₂, . . . , xₚ), the coefficients (b₁, b₂, . . . , bₚ) measure the impact (i.e., the effect size) of covariates, h₀ means the baseline hazard. It corresponds to the value of the hazard if all the xi are equal to zero (the quantity exp(0) equals 1). The t in h(t) reminds us that the hazard may vary over time. A hazard ratio above 1 indicates a covariate that is positively associated with the event probability, and thus negatively associated with the length of survival. In summary, HR=1 means No effect, HR<1 means Reduction in the hazard, HR >1 means Increase in Hazard.

The clinical manifestation database consists of samples from 418 patients. The genetic database comprises data from 469 samples at 1,090,399 loci (with Sequence depth greater than 20 at each locus). Subsequently, data with exonic were filtered, and data matching the names of 89 candidate gene loci were simultaneously extracted. There are 514 gene loci across 469 patients on 89 candidate gene loci after filtering. Due to some clinical manifestation variables having duplicate measurements of inflammatory markers, the database of clinical manifestation variables, gene loci, and inflammatory markers were integrated and cleaned. The final analysis database comprises 2,162 records from 328 patients.

TABLE 1

| | | Set | |
|---|---|---|---|
| | Overall, | training, | testing, |
| Characteristic | N = 328 | N = 170 | N = 158 |
| Gender | | | |
| Male | 181(55.18%) | 92(54.12%) | 89(56.33%) |
| Female | 147(44.82%) | 78(45.88%) | 69(43.67%) |
| Age | 65.00(10.83) | 65.00(11.09) | 66.00(10.47) |
| Survival | | | |
| Survival | 194(59.15%) | 83(48.82%) | 111(70.25%) |
| Dead | 134(40.85%) | 87(51.18%) | 47(29.75%) |
| Received | | | |
| chemotherapy | | | |
| No | 13(3.96%) | 7(4.12%) | 6(3.80%) |
| Yes | 315(96.04%) | 163(95.88%) | 152(96.20%) |
| Received | | | |
| radiation | | | |
| therapy | | | |
| No | 289(88.11%) | 143(84.12%) | 146(92.41%) |
| Yes | 39(11.89%) | 27(15.88%) | 12(7.59%) |
| Received | | | |
| hormone | | | |
| therapy | | | |
| No | 296(90.24%) | 149(87.65%) | 147(93.04%) |
| Yes | 32(9.76%) | 21(12.35%) | 11(6.96%) |
| Received | | | |
| immunotherapy | | | |
| No | 303(92.38%) | 158(92.94%) | 145(91.77%) |
| Yes | 25(7.62%) | 12(7.06%) | 13(8.23%) |
| Received | | | |
| targeted | | | |
| therapy | | | |
| No | 311(94.82%) | 162(95.29%) | 149(94.30%) |
| Yes | 17(5.18%) | 8(4.71%) | 9(5.70%) |
| received | | | |
| surgery | | | |
| No | 215(65.55%) | 104(61.18%) | 111(70.25%) |
| Yes | 113(34.45%) | 66(38.82%) | 47(29.75%) |
| numbers of | 12.00(9.41) | 15.00(10.59) | 11.00(7.63) |
| chemotherapy | | | |

According to the relationship between clinical manifestation variables and survival as follow. Due to BMI, MoCA_TP, MoCA_pass, PHQ_TP, and PHQ_pass involving in duplicate measurements, and these variables may change over time, analysis conducts by using the Cox Proportional Hazard Model with time-varying covariates. And the other variables were analyzed by using the Cox Proportional Hazard Model. The results are presented in Table 2.

TABLE 2

| | Estimate | Std. Error | Z | p-value | HR | 95% CI |
|---|---|---|---|---|---|---|
| Gender (Male) | 0.1296 | 0.2163 | 0.5993 | 0.5490 | 1.14 | (0.75, 1.74) |
| Age | 0.0098 | 0.0099 | 0.9914 | 0.3215 | 1.01 | (0.99, 1.03) |
| Received | −0.0261 | 0.5877 | −0.0444 | 0.9646 | 0.97 | (0.31, 3.08) |
| chemotherapy (Yes) | | | | | | |
| Received radiation | −0.2714 | 0.3009 | −0.9022 | 0.3669 | 0.76 | (0.42, 1.37) |
| therapy (Yes) | | | | | | |
| Received hormone | −0.1250 | 0.3229 | −0.3872 | 0.6986 | 0.88 | (0.47, 1.66) |
| therapy (Yes) | | | | | | |
| Received | −0.4538 | 0.4609 | −0.9846 | 0.3248 | 0.64 | (0.26, 1.57) |
| immunotherapy | | | | | | |
| (Yes) | | | | | | |
| Received targeted | 0.1216 | 0.4609 | 0.2639 | 0.7919 | 1.13 | (0.46, 2.79) |
| therapy (Yes) | | | | | | |
| Received | −1.9111 | 0.3046 | −6.2750 | <0.001*** | 0.15 | (0.08, 0.27) |
| surgery(Yes) | | | | | | |
| BMI | −0.0710 | 0.0324 | −2.1917 | 0.0284* | 0.93 | (0.87, 0.99) |
| Pulse | −0.0107 | 0.0079 | −1.3658 | 0.1720 | 0.99 | (0.97, 1.00) |
| Systolic pressure | −0.0128 | 0.0064 | −2.0083 | 0.0446* | 0.99 | (0.98, 1.00) |
| Diastolic pressure | 0.0468 | 0.0073 | 6.3844 | <0.001*** | 1.05 | (1.03, 1.06) |
| MoCA_TP | −0.0444 | 0.0162 | −2.7344 | 0.0063** | 0.96 | (0.93, 0.99) |
| MoCA_ Pass | 1.0540 | 0.2706 | 3.8957 | <0.001*** | 2.87 | (1.69, 4.88) |
| PHQ_TP | 0.0676 | 0.0194 | 3.4788 | <0.001*** | 1.07 | (1.03, 1.11) |
| PHQ_Pass | 0.6641 | 0.2314 | 2.8704 | 0.0041** | 1.94 | (1.23, 3.06) |

According to the aforementioned univariable analysis model, the clinical manifestation variables of received surgery, BMI, systolic pressure, diastolic pressure, MoCA_TP, MoCA_Pass, PHQ_TP, PHQ_Pass is statistically significant with survival.

The results of model are illustrated as followed. Under the condition of fixing other variables, “received surgery” decreases the risk of death by 85% (HR=0.15) compared with “without received surgery”, and it has a statistically significant impact on survival. Under the condition of fixing other variables, each unit increasing in BMI decreases the risk of death by 7% (HR=0.93), and it has a statistically significant impact on survival. Under the condition of fixing other variables, each unit increasing in systolic pressure decreases the risk of death by 1% (HR=0.99), and it has a statistically significant impact on survival. Under the condition of fixing other variables, each unit increasing in diastolic pressure increases the risk of death by 5% (HR=1.05), and it has a statistically significant impact on survival. Under the condition of fixing other variables, each unit increasing in MoCA_TP decreases the risk of death by 4% (HR=0.96), and it has a statistically significant impact on survival. Under the condition of fixing other variables, patients who did not pass MoCA have approximately 2.87 times higher risk of death (HR=2.87) compared with those who passed, and it has a statistically significant impact on survival. Under the condition of fixing other variables, each unit increasing in PHQ_TP increases the risk of death by 7% (HR=1.07), and it has a statistically significant impact on survival. Under the condition of fixing other variables, patients who did not pass PHQ have approximately 1.94 times higher risk of death (HR=1.94) compared with those who passed, and it has a statistically significant impact on survival.

According to the multivariable analysis model, the variables that were significant in the previous univariable analysis were included in the model. Since the variables MoCA_Pass and PHQ_Pass are classified based on the variables MoCA_TP and PHQ_TP, respectively, the variables MoCA_TP and PHQ_TP were initially used as representatives. As variables from the univariable analysis were time-varying factors, the analysis was conducted using the Cox Proportional Hazard Model with time-varying covariates, and the results are presented in Table 3.

TABLE 3

| | Estimate | Std. Error | Z | p-value | HR | 95% CI |
|---|---|---|---|---|---|---|
| Received surgery | −1.7568 | 0.3111 | −5.6475 | <0.001*** | 0.17 | (0.09, 0.32) |
| (Yes) | | | | | | |
| BMI | −0.0156 | 0.0322 | −0.4859 | 0.6271 | 0.98 | (0.92, 1.05) |
| systolic pressure | −0.0145 | 0.0070 | −2.0607 | 0.0393* | 0.99 | (0.97, 1.00) |
| diastolic pressure | 0.0483 | 0.0085 | 5.6674 | <0.001*** | 1.05 | (1.03, 1.07) |
| MoCA_TP | −0.0217 | 0.0172 | −1.2666 | 0.2053 | 0.98 | (0.95, 1.01) |
| PHQ_TP | 0.0465 | 0.0197 | 2.3581 | 0.0184* | 1.05 | (1.01, 1.09) |

Based on the aforementioned multivariable analysis model, variables such as BMI and MoCA_TP, which is statistically nonsignificant with survival, were progressively eliminated to obtain the most appropriate model reference parameter. The results are presented in Table 4.

TABLE 4

| | Estimate | Std. Error | Z | p-value | HR | 95% CI |
|---|---|---|---|---|---|---|
| Received surgery | −1.8219 | 0.3013 | −6.0458 | <0.001*** | 0.16 | (0.09, 0.29) |
| (Yes) | | | | | | |
| systolic pressure | −0.0150 | 0.0067 | −2.2392 | 0.0251* | 0.99 | (0.97, 1.00) |
| diastolic pressure | 0.0494 | 0.0083 | 5.9176 | <0.001*** | 1.05 | (1.03, 1.07) |
| PHQ_TP | 0.0459 | 0.0198 | 2.3188 | 0.0204* | 1.05 | (1.01, 1.09) |

According to the aforementioned multivariable analysis model, the clinical manifestation variables of received surgery, systolic pressure, diastolic pressure and PHQ_TP, are statistically significant with survival.

The results of model are illustrated as followed. Under the condition of fixing other variables, “received surgery” decreases the risk of death by 84% (HR=0.16) compared with “without received surgery”, and it has a statistically significant impact on survival. Under the condition of fixing other variables, each unit increasing in systolic pressure decreases the risk of death by 1% (HR=0.99), and it has a statistically significant impact on survival. Under the condition of fixing other variables, each unit increasing in diastolic pressure increases the risk of death by 5% (HR=1.05), and it has a statistically significant impact on survival. Under the condition of fixing other variables, each unit increasing in PHQ_TP increases the risk of death by 5% (HR=1.05), and it has a statistically significant impact on survival.

According to the aforementioned data, the relationship statistics between clinical manifestation and survival are as shown in Table 5.

TABLE 5

| | Cox Proportional Hazard Model | | | | | |
|---|---|---|---|---|---|---|
| | Univariable | | | Multivariable | | |
| | HR | 95% CI | p-value | HR | 95% CI | p-value |
| Baseline factors | | | | | | |
| Gender (Male) | 1.14 | (0.75, 1.74) | 0.5490 | — | — | — |
| Age | 1.01 | (0.99, 1.03) | 0.3215 | — | — | — |
| Received | 0.97 | (0.31, 3.08) | 0.9646 | — | — | — |
| chemotherapy (Yes) | | | | | | |
| Received radiation | 0.76 | (0.42, 1.37) | 0.3669 | — | — | — |
| therapy (Yes) | | | | | | |
| Received hormone | 0.88 | (0.47, 1.66) | 0.6986 | — | — | — |
| therapy (Yes) | | | | | | |
| Received | 0.64 | (0.26, 1.57) | 0.3248 | — | — | — |
| immunotherapy | | | | | | |
| (Yes) | | | | | | |
| Received targeted | 1.13 | (0.46, 2.79) | 0.7919 | — | — | — |
| therapy (Yes) | | | | | | |
| Received surgery | 0.15 | (0.08, 0.27) | <0.001*** | 0.16 | (0.09, 0.29) | <0.001*** |
| (Yes) | | | | | | |
| Time-varying | | | | | | |
| factors | | | | | | |
| BMI | 0.93 | (0.87, 0.99) | 0.0284* | — | — | — |
| Pulse | 0.99 | (0.97, 1.00) | 0.1720 | — | — | — |
| Systolic pressure | 0.99 | (0.98, 1.00) | 0.0446* | 0.99 | (0.97, 1.00) | 0.0251* |
| Diastolic pressure | 1.05 | (1.03, 1.06) | <0.001*** | 1.05 | (1.03, 1.07) | <0.001*** |
| MoCA_TP | 0.96 | (0.93, 0.99) | 0.0063** | — | — | — |
| MoCA_ Pass | 2.87 | (1.69, 4.88) | <0.001*** | — | — | — |
| PHQ_TP | 1.07 | (1.03, 1.11) | <0.001*** | 1.05 | (1.01, 1.09) | 0.0204* |
| PHQ_Pass | 1.94 | (1.23, 3.06) | 0.0041** | — | — | — |

According to the aforementioned multivariable analysis model, the clinical manifestation variables of received surgery, systolic pressure, diastolic pressure and PHQ_TP, are statistically significant with survival. Under the condition of fixing other variables, “received surgery” decreases the risk of death by 84% (HR=0.16) compared with “without received surgery”, each unit increasing in systolic pressure decreases the risk of death by 1% (HR=0.99), each unit increasing in diastolic pressure increases the risk of death by 5% (HR=1.05), each unit increasing in PHQ_TP increases the risk of death by 5% (HR=1.05).

According to the relationship between gene loci and survival as follow. Gene loci, selected by LASSO regularization regression, are established the estimation of prognostic indications of pancreatic cancer patient by using Cox Proportional Hazard Model to discuss the relationship between each gene locus and survival. Additionally, four gene loci integrated into the Genetic Risk Score (GRS) and used as variable by the Cox Proportional Hazards Model with time-varying covariates to explore the relationship between genetic loci and survival. The results of Univariable Analysis are shown in Table 6, and the results of Multivariable Analysis are shown in Table 7.

TABLE 6

| | Esti- | Std. | | | | |
|---|---|---|---|---|---|---|
| | mate | Error | Z | p-value | HR | 95% CI |
| rs2071171 | 0.6248 | 0.2322 | 2.6908 | 0.0071** | 1.87 | (1.18, 2.94) |
| rs4806909 | 0.5024 | 0.2261 | 2.2214 | 0.0263* | 1.65 | (1.06, 2.57) |
| rs1042113 | 0.6021 | 0.2261 | 2.6633 | 0.0077** | 1.83 | (1.17, 2.84) |
| rs19211 | 0.3879 | 0.2151 | 1.8036 | 0.0713 | 1.47 | (0.97, 2.25) |
| GRS | 0.9371 | 0.1502 | 6.2407 | <0.001*** | 2.55 | (1.90, 3.43) |

TABLE 7

| | | Std. | | | | |
|---|---|---|---|---|---|---|
| | Estimate | Error | Z | p-value | HR | 95% CI |
| rs2071171 | 0.5863 | 0.2344 | 2.5010 | 0.0124* | 1.80 | (1.14, 2.85) |
| rs4806909 | 0.4643 | 0.2287 | 2.0303 | 0.0423* | 1.59 | (1.02, 2.49) |
| rs1042113 | 0.4395 | 0.2302 | 1.9092 | 0.0562 | 1.55 | (0.99, 2.44) |
| rs19211 | 0.3345 | 0.2155 | 1.5522 | 0.1206 | 1.40 | (0.92, 2.13) |

The variables of statistically significant gene loci from the aforementioned univariable analysis model in Table 6 are as follows. Patients with variant rs2071171 increases risk of death by 1.87 times compared (HR=1.87) compared with without variant, and it has a statistically significant impact on survival. Patients with variant rs4806909 increases risk of death by 1.65 times (HR=1.65) compared with without variant, and it has a statistically significant impact on survival. Patients with variant rs1042113 increases risk of death by 1.83 times (HR=1.83) compared with without variant, and it has a statistically significant impact on survival. When considering the information from all four loci to integrate into the Genetic Risk Score (GRS), each unit increasing in GRS increases risk of death by 2.55 times (HR=2.55), and it has a statistically significant impact on survival.

According to the Table 7, the multivariable analysis model indicates that when simultaneously integrating all four gene loci, both rs2071171 and rs4806909 exhibit statistical significance regarding survival. The model is illustrated as followed. Under the condition of fixing other variables, patients with the variant rs2071171 increases risk of death by 1.80 times (HR=1.80) compared with without variant. Under the condition of fixing other variables, patients with the variant rs4806909 increases risk of death by 1.59 times (HR=1.59) compared with without variant.

According to the aforementioned data, the relationship statistics between gene loci and survival are as shown in Table 8.

TABLE 8

| | Cox Proportional Hazard Model | | | | | |
|---|---|---|---|---|---|---|
| Time-independent | Univariable | | | Multivariable | | |
| factors | HR | 95% CI | p-value | HR | 95% CI | p-value |
| rs2071171 | 1.87 | (1.18, 2.94) | 0.0071** | 1.80 | (1.14, 2.85) | 0.0124* |
| rs4806909 | 1.65 | (1.06, 2.57) | 0.0263* | 1.59 | (1.02, 2.49) | 0.0423* |
| rs1042113 | 1.83 | (1.17, 2.84) | 0.0077** | 1.55 | (0.99, 2.44) | 0.0562 |
| rs1921 | 1.47 | (0.97, 2.25) | 0.0713 | 1.40 | (0.92, 2.13) | 0.1206 |
| GRS | 2.55 | (1.90, 3.43) | <0.001*** | | | |

The relationship statistics between expression level of inflammatory markers and survival are as shown in Table 9. Due to the expression level of inflammatory marker is considered a time-varying factor, analysis will be conducted using the Cox Proportional Hazard Model with time-varying covariates. The results of the univariable analysis are presented in Table 9, while the results of the multivariable analysis are shown in Tables 10 and Tables 11.

The inflammatory marker comprise albumin (ALBUMIN), Creatinine (CREA), C-Reactive Protein (CRP), High-Sensitivity Troponin (hs-cTnT), N terminal pro B type natriuretic peptide (NT-ProBNP), Platelet (Plt), segment (Seg), white blood cell (WBC), Lymphocyte (Lymph), neutrophil-to-lymphocyte ratio (NLR), platelet-to-lymphocyte ratio (PLR), C-reactive protein/albumin ratio (CAR), Carbohydrate antigen 19-9 (CA-199), “hemoglobin, albumin, lymphocyte, and platelet” score (HALP).

TABLE 9

| | Estimate | Std. Error | Z | p-value | HR | 95% CI |
|---|---|---|---|---|---|---|
| ALBUMIN | −1.19497 | 0.12489 | −9.5680 | <0.001*** | 0.30 | (0.240, 0.390) |
| CREA | −0.01186 | 0.00658 | −1.8015 | 0.0716 | 0.99 | (0.980, 1.000) |
| CRP | 0.00976 | 0.00125 | 7.7780 | <0.001*** | 1.01 | (1.010, 1.010) |
| hs-cTnT | 0.00045 | 0.00008 | 5.9239 | <0.001*** | 1.00 | (1.000, 1.000) |
| NTproBNP | 0.00005 | 0.00001 | 6.2530 | <0.001*** | 1.00 | (1.000, 1.000) |
| Plt | −0.00459 | 0.00157 | −2.9299 | 0.0034** | 1.00 | (0.990, 1.000) |
| Seg | 0.06901 | 0.01217 | 5.6701 | <0.001*** | 1.07 | (1.050, 1.100) |
| WBC | 0.11820 | 0.01224 | 9.6591 | <0.001*** | 1.13 | (1.100, 1.150) |
| Lymph | −0.11543 | 0.01660 | −6.9528 | <0.001*** | 0.89 | (0.860, 0.920) |
| NLR | 0.02416 | 0.00533 | 4.5338 | <0.001*** | 1.02 | (1.010, 1.040) |
| PLR | 0.00090 | 0.00039 | 2.3375 | 0.0194* | 1.00 | (1.000, 1.000) |
| CAR | 0.22602 | 0.02465 | 9.1680 | <0.001*** | 1.25 | (1.190, 1.320) |
| CA-199 | 0.00021 | 0.00003 | 8.0987 | <0.001*** | 1.00 | (1.000, 1.000) |
| HALP | −0.01408 | 0.01129 | −1.2474 | 0.2123 | 0.99 | (0.960, 1.010) |

According to the aforementioned univariable analysis model shown in Table 9, ALBUMIN, CRP, NTproBNP, Plt, Seg, WBC, Lymph, NLR, PLR, CAR, and CA-199, are statistically significant with survival.

The results of model are illustrated as followed. Under the condition of fixing other variables, each unit increasing in ALBUMIN decreases the risk of death by 70% (HR=0.30), and it has a statistically significant impact on survival. Under the condition of fixing other variables, each unit increasing in CRP increases the risk of death by 1% (HR=1.01), and it has a statistically significant impact on survival. Under the condition of fixing other variables, each unit increasing in NTproBNP leads to a slight increase in the risk of death (HR=1.00003), and it has a statistically significant impact on survival. Under the condition of fixing other variables, each unit increasing in Plt leads to a slight decrease in the risk of death (HR=0.996), and it has a statistically significant impact on survival. Under the condition of fixing other variables, each unit increasing in Seg increases the risk of death by 7% (HR=1.07), and it has a statistically significant impact on survival. Under the condition of fixing other variables, each unit increasing in WBC increases the risk of death by 13% (HR=1.13), and it has a statistically significant impact on survival. Under the condition of fixing other variables, each unit increasing in Lymph decreases the risk of death by 11% (HR=0.89), and it has a statistically significant impact on survival. Under the condition of fixing other variables, each unit increasing in NLR increases the risk of death by 2% (HR=1.02), and it has a statistically significant impact on survival. Under the condition of fixing other variables, each unit increasing in PLR leads to a slight increase in the risk of death (HR=1.00094), and it has a statistically significant impact on survival. Under the condition of fixing other variables, each unit increasing in CAR increases the risk of death by 25% (HR=1.25), and it has a statistically significant impact on survival. Under the condition of fixing other variables, each unit increasing in CA-199 leads to a slight increase in the risk of death (HR=1.0002), and it has a statistically significant impact on survival.

According to the multivariable analysis model, the variables that were significant in the previous univariable analysis in Table 9 were included in the multivariable analysis model, moreover, analysis was similarly conducted using the Cox Proportional Hazard Model with time-varying covariates.

TABLE 10

| | Estimate | Std. Error | Z | p-value | HR | 95% CI |
|---|---|---|---|---|---|---|
| ALBUMIN | −0.7636 | 0.17431 | −4.3805 | <0.001*** | 0.47 | (0.33, 0.66) |
| CRP | 0.0050 | 0.00136 | 3.6519 | <0.001*** | 1.00 | (1.00, 1.01) |
| hs-cTnT | 0.0000 | 0.00010 | 0.0281 | 0.9776 | 1.00 | (1.00, 1.00) |
| NTproBNP | 0.0000 | 0.00001 | 1.1156 | 0.2646 | 1.00 | (1.00, 1.00) |
| Plt | −0.0041 | 0.00115 | −3.5839 | <0.001*** | 1.00 | (0.99, 1.00) |
| WBC | 0.0745 | 0.01293 | 5.7585 | <0.001*** | 1.08 | (1.05, 1.10) |
| PLR | 0.0006 | 0.00045 | 1.4297 | 0.1528 | 1.00 | (1.00, 1.00) |
| CA-199 | 0.0001 | 0.00003 | 3.2860 | 0.0010* | 1.00 | (1.00, 1.00) |

Based on the aforementioned multivariable analysis model in Table 10, further elimination of nonsignificant variables was performed to obtain more suitable parameter estimates, as shown in Table 11.

TABLE 11

| | Estimate | Std. Error | Z | p-value | HR | 95% CI |
|---|---|---|---|---|---|---|
| ALBUMIN | −0.80940 | 0.15116 | −5.3545 | <0.001*** | 0.45 | (0.33, 0.60) |
| CRP | 0.00509 | 0.00139 | 3.6672 | <0.001*** | 1.01 | (1.00, 1.01) |
| Plt | −0.00368 | 0.00114 | −3.2379 | 0.0012** | 1.00 | (0.99, 1.00) |
| WBC | 0.07645 | 0.01270 | 6.0220 | <0.001*** | 1.08 | (1.05, 1.11) |
| CA-199 | 0.00010 | 0.00003 | 3.4151 | <0.001*** | 1.00 | (1.00, 1.00) |

According to the aforementioned multivariable analysis model, the ALBUMIN, CRP, Plt, WBC, and CA-199 are statistically significant with survival.

The results of model are illustrated as followed. Under the condition of fixing other variables, each unit increasing in ALBUMIN decreases the risk of death by 55% (HR=0.45), and it has a statistically significant impact on survival. Under the condition of fixing other variables, each unit increasing in CRP increases the risk of death by 1% (HR=1.01), and it has a statistically significant impact on survival. Under the condition of fixing other variables, each unit increasing in Plt leads to a slight decrease in the risk of death (HR=0.996), and it has a statistically significant impact on survival. Under the condition of fixing other variables, each unit increasing in WBC increases the risk of death by 8% (HR=1.08), and it has a statistically significant impact on survival. Under the condition of fixing other variables, each unit increasing in CA-199 leads to a slight increase in the risk of death (HR=1.0001), and it has a statistically significant impact on survival.

According to the aforementioned data, the relationship statistics between expression level of inflammatory marker and survival are as shown in Table 12.

TABLE 12

| | Cox Proportional Hazard Model | | | | | |
|---|---|---|---|---|---|---|
| Time-varying | Univariable | | | Multivariable | | |
| factors | HR | 95% CI | p-value | HR | 95% CI | p-value |
| ALBUMIN | 0.30 | (0.240, 0.390) | <0.001*** | 0.45 | (0.33, 0.60) | <0.001*** |
| CREA | 0.99 | (0.980, 1.000) | 0.0716 | — | — | — |
| CRP | 1.01 | (1.010, 1.010) | <0.001*** | 1.01 | (1.00, 1.01) | <0.001*** |
| hscTnT | 1.00 | (1.000, 1.000) | <0.001*** | — | — | — |
| NTproBNP | 1.00 | (1.000, 1.000) | <0.001*** | — | — | — |
| Plt | 1.00 | (0.990, 1.000) | 0.0034** | 1.00 | (0.99, 1.00) | 0.0012** |
| Seg | 1.07 | (1.050, 1.100) | <0.001*** | — | — | — |
| WBC | 1.13 | (1.100, 1.150) | <0.001*** | 1.08 | (1.05, 1.11) | <0.001*** |
| Lymph | 0.89 | (0.860, 0.920) | <0.001*** | — | — | — |
| NLR | 1.02 | (1.010, 1.040) | <0.001*** | — | — | — |
| PLR | 1.00 | (1.000, 1.000) | 0.0194* | — | — | — |
| CAR | 1.25 | (1.190, 1.320) | <0.001*** | — | — | — |
| CA-199 | 1.00 | (1.000, 1.000) | <0.001*** | 1.00 | (1.00, 1.00) | <0.001*** |
| HALP | 0.99 | (0.960, 1.010) | 0.2123 | — | — | — |

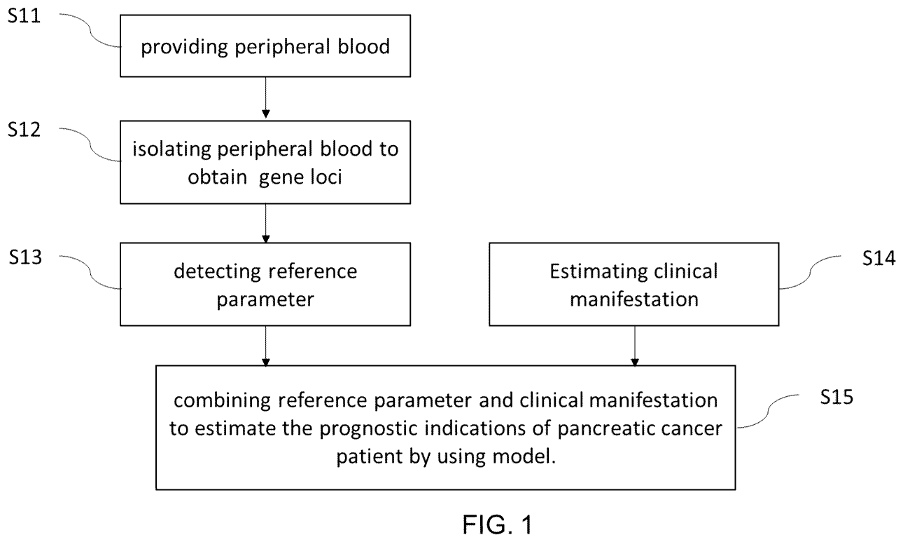

Please refer to FIG. 1. FIG. 1 is a flowing diagram illustrating method for predicting a prognostic indications of a pancreatic cancer patients extract according to the present invention. As shown in FIG. 1, the steps of method for predicting a prognostic indications of a pancreatic cancer patients includes step S11 to step S15.

Step S11: providing peripheral blood. In step S11, the sample is taken out from peripheral blood. The peripheral blood is the flowing, circulating blood of the body. It is composed of blood cells, blood plasma. The blood cells are suspended in blood plasma, through which the blood cells are circulated through the body.

Step S12: isolating peripheral blood to obtain gene locus. In this step, the method is used to isolate gen loci from peripheral blood includes the process of RNA Extraction, DNA Extraction, Polymerase Chain Reaction (PCR), Reverse Transcription Polymerase Chain Reaction (RT-PCR).

Step S13: detecting reference parameter. In this step, at least one single nucleotide polymorphism of gene loci is detected. In this embodiment, the detecting method includes Polymorphism Analysis, Gene Locus Sequencing, Gene Locus Microarray, Linkage Disequilibrium Analysis, Methylation Analysis.

Step S14: estimating clinical manifestations. In this step, at least one clinical manifestation is estimated. The clinical manifestations might be gender, age, received chemotherapy, received radiation therapy, received hormone therapy, received immunotherapy, received targeted therapy, received surgery, tumor stage, body mass index (BMI), systolic pressure, diastolic pressure, Montreal Cognitive Assessment (MoCA), 9-item self-report Patient Health Questionnaire (PHQ-9). In some embodiment, the clinical manifestations are received surgery, systolic pressure, diastolic pressure and 9-item self-report Patient Health Questionnaire (PHQ-9).

In should be explained that when step S14 is executed, the step S13 and step S14 might be executed at the same time. In some embodiments, step S13 and step S14 are optional.

Step S15: combining reference parameter and clinical manifestation to estimate the prognostic indications of pancreatic cancer patient by using model. In this step, the prognostic indication is estimated by using a specific model to calculate and predict the prognostic indications of a pancreatic cancer patients, in which the special model includes Genetic Risk Score, Univariable Analysis, Multivariable Analysis, Dominant Model, Least Absolute Shrinkage and Selection Operator, Kaplan-Meier Curve, and Cox Proportional Hazard Model.

According to the Multivariable Analysis results of clinical variables, after 4 gene loci are individually added to the Cox Proportional Hazard Model with time varying covariates analysis, the results are as listed in Table 13. Received surgery, systolic pressure, diastolic pressure, PHQ_TP is statistically significant with the gene locus rs2071171.

TABLE 13

| | Estimate | Std. Error | Z | p-value | HR | 95% CI |
|---|---|---|---|---|---|---|
| Received surgery | −1.7819 | 0.3025 | −5.8898 | <0.001**** | 0.17 | (0.09, 0.30) |
| (Yes) | | | | | | |
| systolic pressure | −0.0157 | 0.0066 | −2.3754 | 0.0175* | 0.98 | (0.97, 1.00) |
| diastolic pressure | 0.0499 | 0.0082 | 6.0535 | <0.001*** | 1.05 | (1.03, 1.07) |
| PHQ_TP | 0.0448 | 0.0195 | 2.2933 | 0.0218* | 1.05 | (1.01, 1.09) |
| rs2071171 | 0.5172 | 0.2123 | 2.4362 | 0.0148* | 1.68 | (1.11, 2.54) |

According to the aforementioned multivariable analysis model, received surgery, systolic pressure, diastolic pressure, PHQ_TP, and rs2071171 are all statistically significant with survival. The model is illustrated as followed. Under the condition of fixing other variables, “received surgery” decreases the risk of death by 83% (HR=0.17) compared with “without received surgery”, and it has a statistically significant impact on survival. Under the condition of fixing other variables, each unit increasing in systolic pressure decreases the risk of death by 2% (HR=0.98), and it has a statistically significant impact on survival. Under the condition of fixing other variables, each unit increasing in diastolic pressure increases the risk of death by 5% (HR=1.05), and it has a statistically significant impact on survival. Under the condition of fixing other variables, each unit increasing in PHQ_TP increases the risk of death by 5% (HR=1.05), and it has a statistically significant impact on survival. Under the condition of fixing other variables, with variation in rs2071171 increases the risk of death by 68% (HR=1.68) compared with without variation in rs2071171, and it has a statistically significant impact on survival.

According to the aforementioned multivariable analysis model, after 4 gene loci summarized in Genetic Risk Score and added to the Cox Proportional Hazard Model with time varying covariates analysis, the results are as listed in Table 14. Received surgery, systolic pressure, diastolic pressure, PHQ_TP is statistically significant with the GRS results.

TABLE 14

| | Estimate | Std. Error | Z | p-value | HR | 95% CI |
|---|---|---|---|---|---|---|
| Received surgery | −1.6194 | 0.3056 | −5.2997 | <0.001*** | 0.20 | (0.11, 0.36) |
| (Yes) | | | | | | |
| systolic pressure | −0.0144 | 0.0062 | −2.3112 | 0.0208* | 0.99 | (0.97, 1.00) |
| diastolic pressure | 0.0474 | 0.0082 | 5.7879 | <0.001*** | 1.05 | (1.03, 1.07) |
| PHQ_TP | 0.0403 | 0.0199 | 2.0308 | 0.0423* | 1.04 | (1.00, 1.08) |
| GRS | 0.6603 | 0.1418 | 4.6563 | <0.001*** | 1.94 | (1.47, 2.56) |

According to the aforementioned multivariable analysis model, received surgery, systolic pressure, diastolic pressure, PHQ_TP, and GRS are all statistically significant for survival. The model is illustrated as followed. Under the condition of fixing other variables, “received surgery” decreases the risk of death by 80% (HR=0.20) compared with “without received surgery”, and it has a statistically significant impact on survival. Under the condition of fixing other variables, each unit increasing in systolic pressure decreases the risk of death by 1% (HR=0.99), and it has a statistically significant impact on survival. Under the condition of fixing other variables, each unit increasing in diastolic pressure increases the risk of death by 5% (HR=1.05), and it has a statistically significant impact on survival. Under the condition of fixing other variables, each unit increasing in PHQ_TP increases the risk of death by 4% (HR=1.04), and it has a statistically significant impact on survival. Under the condition of fixing other variables, each unit increasing in GRS increases the risk of death by 94% (HR=1.94), and it has a statistically significant impact on survival.

According to the aforementioned data, the relationship statistics between clinical manifestation, gene loci and survival are as shown in Table 15.

TABLE 15

| | Cox Proportional Hazard Model | | | | | | | | |
|---|---|---|---|---|---|---|---|---|---|
| | Univariable | | | Multivariable | | | Multivariable | | |
| | HR | 95% CI | p-value | HR | 95% CI | p-value | HR | 95% CI | p-value |
| clinical | | | | | | | | | |
| manifestation | | | | | | | | | |
| Received | 0.15 | (0.08, | <0.001*** | 0.17 | (0.09, | <0.001*** | 0.20 | (0.11, | <0.001*** |
| surgery (Yes) | | 0.27) | | | 0.30) | | | 0.36) | |
| systolic | 0.99 | (0.98, | 0.0446* | 0.98 | (0.97, | 0.0175* | 0.99 | (0.97, | 0.0208* |
| pressure | | 1.00) | | | 1.00) | | | 1.00) | |
| diastolic | 1.05 | (1.03, | <0.001*** | 1.05 | (1.03, | <0.001*** | 1.05 | (1.03, | <0.001*** |
| pressure | | 1.06) | | | 1.07) | | | 1.07) | |
| PHQ_TP | 1.07 | (1.03, | <0.001*** | 1.05 | (1.01, | 0.0218* | 1.04 | (1.00, | 0.0423* |
| | | 1.11) | | | 1.09) | | | 1.08) | |
| gene loci | | | | | | | | | |
| rs2071171 | 1.87 | (1.18, | 0.0071** | 1.68 | (1.11, | 0.0148* | — | — | — |
| | | 2.94) | | | 2.54) | | | | |
| GRS | 2.55 | (1.90, | <0.001*** | — | — | — | 1.94 | (1.47, | <0.001*** |
| | | 3.43) | | | | | | 2.56) | |

According to the aforementioned multivariable analysis model, whatever the combination of clinical manifestation of received surgery, systolic pressure, diastolic pressure, PHQ_TP with the gene loci rs2071171 or GRS are all statistically significant with survival.

When the variables include received surgery, systolic pressure, diastolic pressure, PHQ_TP and rs2071171, the model is illustrated as followed. Under the condition of fixing other variables, “received surgery” decreases the risk of death by 83% (HR=0.17) compared with “without received surgery”. Under the condition of fixing other variables, each unit increasing in systolic pressure decreases the risk of death by 2% (HR=0.98). Under the condition of fixing other variables, each unit increasing in diastolic pressure increases the risk of death by 5% (HR=1.05). Under the condition of fixing other variables, each unit increasing in PHQ_TP increases the risk of death by 5% (HR=1.05). Under the condition of fixing other variables, patients with a variation in rs2071171 have a hazard ratio of 1.68 compared to patients without the variation, indicating an increased risk of mortality.

When the variables include received surgery, systolic pressure, diastolic pressure, PHQ_TP and GRS, the model is illustrated as followed.

Under the condition of fixing other variables, “received surgery” decreases the risk of death by 80% (HR=0.20) compared with “without received surgery”. Under the condition of fixing other variables, each unit increasing in systolic pressure decreases the risk of death by 1% (HR=0.99). Under the condition of fixing other variables, each unit increasing in diastolic pressure increases the risk of death by 5% (HR=1.05). Under the condition of fixing other variables, each unit increasing in PHQ_TP increases the risk of death by 4% (HR=1.04). Under the condition of fixing other variables, each unit increasing in GRS corresponds to have a hazard ratio of 1.94, indicating an increased risk of mortality.

In some embodiment, the gene loci selected through the LASSO, specific gene locus beta values at each time stage were estimated by training set data and logistic regression. These beta values were used in the calculation of Genetic Risk Scores (GRS) over time. The Cox Proportional Hazard Model with time and varying covariates explore the relationship between genetic loci and survival. By calculating the two indicators, AIC (Akaike Information Criterion) and BIC (Bayesian Information Criterion), the invention evaluate the complexity of the survival prediction model and the goodness of the model fitting data. The survival prediction model, which simultaneously incorporates specific clinical manifestation variables and GRS, was the optimal predictive model.

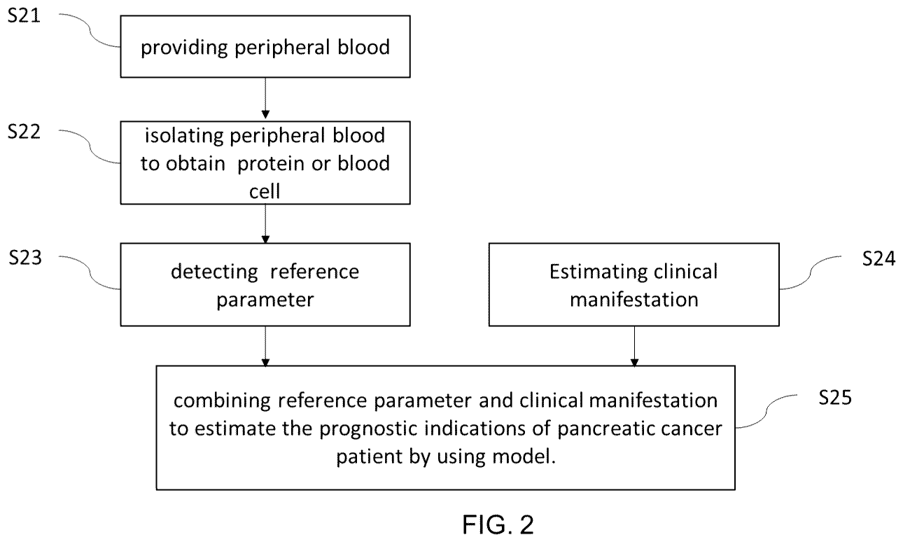

Please refer to FIG. 2. FIG. 2 is a flowing diagram illustrating method for predicting a prognostic indications of a pancreatic cancer patients extract according to the present invention. As shown in FIG. 2, the steps of method for predicting a prognostic indications of a pancreatic cancer patients includes step S21 to step S25.

Step S21: providing peripheral blood. In step S21, the detail process is same as previous step S11, and it is not described repeatedly herein.

Step S22: isolating peripheral blood to obtain protein or blood cell. In this step, the isolating method includes the process of cell lysis, centrifugation, precipitation, proteinase digestion, isolation of subcellular fractions, the process of flow cytometry, hemocytometer, and blood smear.

Step S23: detecting reference parameter. In this step, at least one expression level of inflammatory marker from at least one desired protein expression level or number of blood cells is detected. The detecting method includes blood test, C-Reactive protein test, white blood cell count, neutrophil-to-lymphocyte ratio (NLR), platelet-to-lymphocyte ratio (PLR), advanced liver cancer inflammation index (ALI), serum protein test, cell culture, immunohistochemical staining, molecular biology techniques.

Step S24: estimating clinical manifestations. In step S24, process is same as previous step S14, and it is not described repeatedly herein.

In should be explained that when step S24 is executed, the step S23 and step S24 might be executed at the same time. In some embodiments, step S23 and step S24 are optional.

Step S25: combining reference parameter and clinical manifestation to estimate the prognostic indications of pancreatic cancer patient by using model. In this step, the prognostic indication is estimated by using a specific model to calculate and predict the prognostic indications of a pancreatic cancer patients, in which the special model includes univariable analysis, multivariable analysis and Cox Proportional Hazard Model.

According to the multivariable analysis results of clinical manifestation and expression level of inflammatory marker, analyzed using the Cox Proportional Hazard Model with time-varying covariates. The results are as shown in Table 16.

TABLE 16

| | Estimate | Std. Error | Z | p-value | HR | 95% CI |
|---|---|---|---|---|---|---|
| Received surgery | −1.29642 | 0.3255 | −3.9826 | <0.001*** | 0.27 | (0.14, 0.52) |
| (Yes) | | | | | | |
| systolic pressure | −0.01487 | 0.0066 | −2.2536 | 0.0242* | 0.99 | (0.97, 1.00) |
| diastolic pressure | 0.03847 | 0.0092 | 4.1620 | <0.001*** | 1.04 | (1.02, 1.06) |
| PHQ_TP | 0.01405 | 0.0187 | 0.7516 | 0.4523 | 1.01 | (0.98, 1.05) |
| ALBUMIN | −0.58672 | 0.1434 | −4.0927 | <0.001*** | 0.56 | (0.42, 0.74) |
| CRP | 0.00572 | 0.0014 | 3.9692 | <0.001*** | 1.01 | (1.00, 1.01) |
| Plt | −0.00384 | 0.0012 | −3.1515 | 0.0016** | 1.00 | (0.99, 1.00) |
| WBC | 0.07173 | 0.0121 | 5.9253 | <0.001*** | 1.07 | (1.05, 1.10) |
| CA-199 | 0.00005 | 0.0000 | 1.3401 | 0.1802 | 1.00 | (1.00, 1.00) |

Based on the aforementioned multivariable models, PHQ_TP and CA-199 are statistically nonsignificant with survival. Obviously, received surgery, systolic pressure, diastolic pressure, ALBUMIN, CRP, Plt, WBC is statistically significant with survival. The model is illustrated as followed. Under the condition of fixing other variables, “received surgery” decreases the risk of death by 73% (HR=0.27) compared with “without received surgery”, and it has a statistically significant impact on survival. Under the condition of fixing other variables, each unit increasing in systolic pressure decreases the risk of death by 1% (HR=0.99), and it has a statistically significant impact on survival. Under the condition of fixing other variables, each unit increasing in diastolic pressure increases the risk of death by 4% (HR=1.04), and it has a statistically significant impact on survival. Under the condition of fixing other variables, each unit increasing in ALBUMIN decreases the risk of death by 44% (HR=0.56), and it has a statistically significant impact on survival. Under the condition of fixing other variables, each unit increasing in CRP increases the risk of death by 1% (HR=1.01), and it has a statistically significant impact on survival. Under the condition of fixing other variables, each unit increasing in Plt slightly decreases the risk of death (HR=0.996), and it has a statistically significant impact on survival. Under the condition of fixing other variables, each unit increasing in WBC increases the risk of death by 7% (HR=1.07), and it has a statistically significant impact on survival.

According to the aforementioned data, the relationship statistics between clinical manifestation, expression level of inflammatory marker and survival are as shown in Table 17.

TABLE 17

| | Cox Proportional Hazard Model | | | | | |
|---|---|---|---|---|---|---|
| | Univariable | | | Multivariable | | |
| | HR | 95% CI | p-value | HR | 95% CI | p-value |
| clinical | | | | | | |
| manifestation | | | | | | |
| Received surgery | 0.15 | (0.08, 0.27) | <0.001*** | 0.27 | (0.14, 0.52) | <0.001*** |
| (Yes) | | | | | | |
| systolic pressure | 0.99 | (0.98, 1.00) | 0.0446* | 0.99 | (0.97, 1.00) | 0.0242* |
| diastolic pressure | 1.05 | (1.03, 1.06) | <0.001*** | 1.04 | (1.02, 1.06) | <0.001*** |
| PHQ_TP | 1.07 | (1.03, 1.11) | <0.001*** | 1.01 | (0.98, 1.05) | 0.4523 |
| inflammatory | | | | | | |
| marker | | | | | | |
| ALBUMIN | 0.30 | (0.240, 0.390) | <0.001*** | 0.56 | (0.42, 0.74) | <0.001*** |
| CRP | 1.01 | (1.010, 1.010) | <0.001*** | 1.01 | (1.00, 1.01) | <0.001*** |
| Plt | 1.00 | (0.990, 1.000) | 0.0034** | 1.00 | (0.99, 1.00) | 0.0016** |
| WBC | 1.13 | (1.100, 1.150) | <0.001*** | 1.07 | (1.05, 1.10) | <0.001*** |
| CA-199 | 1.00 | (1.000, 1.000) | <0.001*** | 1.00 | (1.00, 1.00) | 0.1802 |

Based on the aforementioned multivariable models, received surgery, systolic pressure, diastolic pressure, ALBUMIN, CRP, Plt, WBC are statistically significant with survival.

Under the condition of fixing other variables, “received surgery” decreases the risk of death by 73% (HR=0.27) compared with “without received surgery”. Under the condition of fixing other variables, each unit increasing in systolic pressure decreases the risk of death by 1% (HR=0.99). Under the condition of fixing other variables, each unit increasing in diastolic pressure increases the risk of death by 4% (HR=1.04). Under the condition of fixing other variables, each unit increasing in ALBUMIN decreases the risk of death by 44% (HR=0.56). Under the condition of fixing other variables, each unit increasing in CRP increases the risk of death by 1% (HR=1.01). Under the condition of fixing other variables, each unit increasing in Plt slightly decreases the risk of death (HR=0.996). Under the condition of fixing other variables, each unit increasing in WBC increases the risk of death by 7% (HR=1.07).

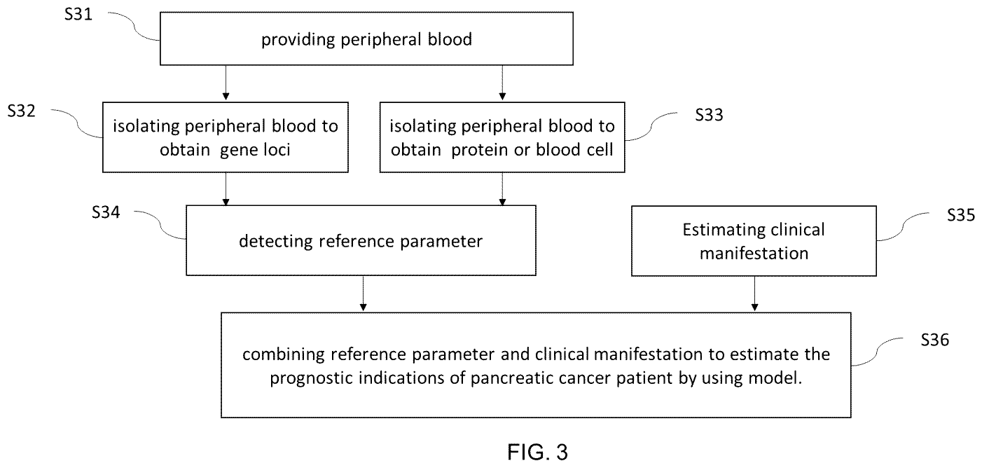

Please refer to FIG. 3. FIG. 3 is a flowing diagram illustrating method for predicting a prognostic indications of a pancreatic cancer patients extract according to the present invention. As shown in FIG. 3, the steps of method for predicting a prognostic indications of a pancreatic cancer patients includes step S31 to step S36.

Step S31: providing peripheral blood. In this step S31, is the detail process is same as previous step S11, and it is not described repeatedly herein.

Step S32: isolating peripheral blood to obtain gene loci. In this step, the detail process is same as previous step S12.

Step S33: isolating peripheral blood to obtain protein or blood cell. In this step, the detail process is same as previous step S22, and it is not described repeatedly herein.

Step S34: detecting reference parameter. In this step, at least one single nucleotide polymorphism of gene loci or expression level of inflammatory marker is detected. The detecting method for single nucleotide polymorphism of gene loci includes polymorphism analysis, gene locus sequencing, gene locus microarray, linkage disequilibrium analysis, methylation analysis, and the detecting method for expression level of inflammatory marker includes blood test, C-Reactive Protein Test, white blood cell count, neutrophil-to-lymphocyte ratio (NLR), platelet-to-lymphocyte ratio (PLR), advanced liver cancer inflammation index (ALI), serum protein test, cell culture, immunohistochemical staining, molecular biology techniques.

Step S35: estimating clinical manifestations. In this step S35, the detail process is same as previous step S14, and it is not described repeatedly herein.

In should be explained that when step S35 is executed, the steps S32, S33 and S34 might be executed at the same time. In some embodiments, the steps S32, S33, S34 and S35 are optional.

Step S36: combining reference parameter and clinical manifestation to estimate the prognostic indications of pancreatic cancer patient by using model. In this step, the prognostic indication is estimated by using a specific model to calculate and predict the prognostic indications of a pancreatic cancer patients, in which the special model includes Genetic

Risk Score, Univariable Analysis, Multivariable Analysis, Dominant Model, Least Absolute Shrinkage and Selection Operator, Kaplan-Meier Curve, and Cox Proportional Hazard Model, Akaike Information Criterion and Bayesian Information Criterion.

According to the results of clinical manifestation, genetic loci, and expression level of inflammatory marker, and survival, which analyzed by using the Cox Proportional Hazard Model with time-varying covariates. The results are as listed in Table 18, clinical manifestation of received surgery, systolic pressure, diastolic pressure, 4 genetic loci and inflammatory markers of ALBUMIN, CRP, Plt, and WBC demonstrate statistical significance simultaneously.

TABLE 18

| | Estimate | Std. Error | Z | p-value | HR | 95% CI |
|---|---|---|---|---|---|---|
| Received surgery | −1.26824 | 0.32873 | −3.8580 | <0.001*** | 0.28 | (0.15, 0.54) |
| (Yes) | | | | | | |
| systolic pressure | −0.01677 | 0.00630 | −2.6633 | 0.0077** | 0.98 | (0.97, 1.00) |
| diastolic pressure | 0.03772 | 0.00907 | 4.1587 | <0.001*** | 1.04 | (1.02, 1.06) |
| GRS | 0.42609 | 0.16226 | 2.6261 | 0.0086** | 1.53 | (1.11, 2.10) |
| ALBUMIN | −0.57767 | 0.14130 | −4.0881 | <0.001*** | 0.56 | (0.43, 0.74) |
| CRP | 0.00603 | 0.00153 | 3.9508 | <0.001*** | 1.01 | (1.00, 1.01) |
| Plt | −0.00358 | 0.00114 | −3.1414 | 0.0017** | 1.00 | (0.99, 1.00) |
| WBC | 0.07158 | 0.01127 | 6.3522 | <0.001*** | 1.07 | (1.05, 1.10) |

Based on the multivariable models, progressively excluding variables PHQ_TP and CA199, which are statistically nonsignificant with survival, to obtain more appropriate model parameter estimates. Obviously, received surgery, systolic pressure, diastolic pressure, GRS, ALBUMIN, CRP, Plt, and WBC is statistically significant with survival. The model is illustrated as followed. Under the condition of fixing other variables, “received surgery” decreases the risk of death by 72% (HR=0.28) compared with “without received surgery”, and it has a statistically significant impact on survival. Under the condition of fixing other variables, each unit increasing in systolic pressure decreases the risk of death by 2% (HR=0.98), and it has a statistically significant impact on survival. Under the condition of fixing other variables, each unit increasing in diastolic pressure increases the risk of death by 4% (HR=1.04), and it has a statistically significant impact on survival. Under the condition of fixing other variables, each unit increasing in GRS increases the risk of death by 53% (HR=1.53), and it has a statistically significant impact on survival. Under the condition of fixing other variables, each unit increasing in ALBUMIN decreases the risk of death by 44% (HR=0.56), and it has a statistically significant impact on survival. Under the condition of fixing other variables, each unit increasing in CRP increases the risk of death by 1% (HR=1.01), and it has a statistically significant impact on survival. Under the condition of fixing other variables, each unit increasing in Plt slightly decreases the risk of death (HR=0.996), and it has a statistically significant impact on survival. Under the condition of fixing other variables, each unit increasing in WBC increases the risk of death by 7% (HR=1.07), and it has a statistically significant impact on survival.

According to the aforementioned data, the relationship statistics between clinical manifestation, gene loci, expression level of inflammatory marker and survival are as shown in Table 19.

TABLE 19

| | Cox Proportional Hazard Model | | | | | |
|---|---|---|---|---|---|---|
| | Univariable | | | Multivariable | | |
| | HR | 95% CI | p-value | HR | 95% CI | p-value |
| clinical | | | | | | |
| manifestation | | | | | | |
| Received surgery | 0.15 | (0.08, 0.27) | <0.001*** | 0.28 | (0.15, 0.54) | <0.001*** |
| (Yes) | | | | | | |
| systolic pressure | 0.99 | (0.98, 1.00) | 0.0446* | 0.98 | (0.97, 1.00) | 0.0077** |
| diastolic pressure | 1.05 | (1.03, 1.06) | <0.001*** | 1.04 | (1.02, 1.06) | <0.001*** |
| gene loci | | | | | | |
| GRS | 2.55 | (1.90, 3.43) | <0.001*** | 1.53 | (1.11, 2.10) | 0.0086** |
| inflammatory | | | | | | |
| marker | | | | | | |
| ALBUMIN | 0.30 | (0.240, 0.390) | <0.001*** | 0.56 | (0.43, 0.74) | <0.001*** |
| CRP | 1.01 | (1.010, 1.010) | <0.001*** | 1.01 | (1.00, 1.01) | <0.001*** |
| Plt | 1.00 | (0.990, 1.000) | 0.0034** | 1.00 | (0.99, 1.00) | 0.0017** |
| WBC | 1.13 | (1.100, 1.150) | <0.001*** | 1.07 | (1.05, 1.10) | <0.001*** |

By calculating the Akaike Information Criterion (AIC) and Bayesian Information Criterion (BIC) to evaluate the complexity of the statistical model, and the quality of data. In general, the model with the smaller AIC or BIC values indicates better model.

TABLE 20

| Model combinations | Variables | AIC | BIC |
|---|---|---|---|
| clinical manifestation | received surgery, systolic pressure, | 716.13 | 726 |
| | diastolic pressure, PHQ_TP | | |
| clinical manifestation | received surgery, systolic pressure, | 712.94 | 725.27 |
| and gene loci | diastolic pressure, PHQ_TP, rs2071171 | | |
| clinical manifestation | received surgery, systolic pressure, | 694.41 | 706.74 |
| and gene loci | diastolic pressure, PHQ_TP, GRS | | |
| clinical manifestation | received surgery, systolic pressure, | 603.18 | 625.37 |
| and inflammatory marker | diastolic pressure, ALBUMIN, CRP, | | |
| | Plt, WBC | | |
| clinical manifestation, | received surgery, systolic pressure, | 595.86 | 615.58 |
| gene loci and | diastolic pressure, GRS, ALBUMIN, | | |
| inflammatory marker | CRP, Plt, WBC | | |

Compared with six model combinations, simultaneously considers clinical manifestation, gene loci and inflammatory marker has the lowest AIC and BIC values, and it indicates the model combinations are better.

The foregoing descriptions are only preferred embodiments of the present invention and are not used to limit the scope of the present invention. Meanwhile, any person with ordinary knowledge in the art can easily understand and implement it. Therefore, any equivalent variation or modification without departing from the spirit of the present invention disclosed herein is to be included within the scope of the present invention.

What is claimed is:

1. A method for predicting a prognostic indications of pancreatic cancer patient, comprising:
providing a sample, wherein the sample is peripheral blood;
isolating the sample to obtain an object, wherein the object is a gene loci;
detecting a reference parameter from the object for estimating a prognostic indicators of pancreatic cancer patient, wherein the reference parameter is a single nucleotide polymorphism of gene loci; and
combining the reference parameter and a clinical manifestation to estimate the prognostic indications of pancreatic cancer patient by using a model.

2. The method for predicting a prognostic indications of pancreatic cancer patient according to claim 1, wherein the peripheral blood includes blood cells and blood plasma.

3. The method for predicting a prognostic indications of pancreatic cancer patient according to claim 1, the method of isolating the sample includes a process of RNA Extraction, a DNA extraction, a polymerase chain reaction (PCR), and a reverse transcription polymerase chain reaction (RT-PCT).

4. The method for predicting a prognostic indications of pancreatic cancer patient according to claim 1, wherein the clinical manifestation includes received surgery, systolic pressure, diastolic pressure, 9-item-report patient health questionnaire (PHQ-9).

5. The method for predicting a prognostic indications of pancreatic cancer patient according to claim 1, wherein detecting method of detecting the reference parameter includes Polymorphism Analysis, Gene Locus Sequencing, Gene Locus Microarray, Gene Locus-Specific PCR, Gene Locus Linkage Disequilibrium Analysis, Gene Locus Methylation Analysis.

6. The method for predicting a prognostic indications of pancreatic cancer patient according to claim 1, wherein the model includes Genetic Risk Score, Univariable Analysis, Multivariable Analysis, Dominant Model, Least Absolute Shrinkage and Selection Operator, Kaplan-Meier Curve, Cox Proportional Hazard Model.

7. The method for predicting a prognostic indications of pancreatic cancer patient according to claim 1, wherein the gene locus includes rs2071171, rs48069091, rs10421131, rs1921.

8. A method for predicting a prognostic indications of pancreatic cancer patient, comprising:
providing a sample, wherein the sample is peripheral blood;
isolating the sample to obtain an object, wherein the object is protein or blood cell;
detecting a reference parameter from the object for estimating a prognostic indicators of pancreatic cancer patient, wherein the reference parameter is an object expression level of an inflammatory marker from the object to obtain at least one desired protein expression level or a number of blood cells; and
combining the reference parameter and a clinical manifestation to estimate the prognostic indications of pancreatic cancer patient by using a model.

9. The method for predicting a prognostic indications of pancreatic cancer patient according to claim 8, wherein the peripheral blood includes blood cells and blood plasma.

10. The method for predicting a prognostic indications of pancreatic cancer patient according to claim 8, wherein when the object is protein, the method of isolating the sample includes a process of cell lysis, centrifugation, precipitation, proteinase digestion, and isolation of subcellular fractions;
wherein when the object is blood cells, the method of isolating the sample includes a process of flow cytometry, hemocytometer, blood smear.

11. The method for predicting a prognostic indications of pancreatic cancer patient according to claim 8, wherein the clinical manifestation includes received surgery, systolic pressure, diastolic pressure, 9-item-report patient health questionnaire (PHQ-9).

12. The method for predicting a prognostic indications of pancreatic cancer patient according to claim 8, wherein the model includes Univariable Analysis, Multivariable Analysis, Cox Proportional Hazard Model.

13. The method for predicting a prognostic indications of pancreatic cancer patient according to claim 8, wherein the inflammatory marker includes at least one of albumin, C-Reactive protein (CRP), platelet (Plt), white blood cell (WBC), and CA199.

14. The method for predicting a prognostic indications of pancreatic cancer patient according to claim 8, wherein detecting method to detect the object expression level of the inflammatory marker includes Blood Test, C-Reactive Protein Test, White Blood Cell Count, Neutrophil-to-Lymphocyte Ratio (NLR), Platelet-to-Lymphocyte Ratio (PLR), Advanced Liver Cancer Inflammation Index (ALI), Serum Protein Test, Cell Culture, Immunohistochemical Staining, and Molecular Biology Techniques.

15. A method for predicting a prognostic indications of pancreatic cancer patient, comprising:
providing a sample, wherein the sample is peripheral blood;
isolating the sample to obtain an object, wherein the object includes a gene locus, a protein or a blood cell;
detecting a reference parameter from the object for estimating a prognostic indicators of pancreatic cancer patient; and
estimating the prognostic indications of pancreatic cancer patient by using a model;
wherein the reference parameter comprises at least one of a single nucleotide polymorphism of gene locus, an object expression level of an inflammatory marker, and a clinical manifestation;
wherein the single nucleotide polymorphism of gene locus is detected from the object to confirm the single nucleotide polymorphism of a desired gene locus;
wherein the object expression level of the inflammatory marker is detected from the object to obtain at least one desired protein expression level or a number of blood cells.

16. The method for predicting a prognostic indications of pancreatic cancer patient according to claim 15, wherein the peripheral blood includes blood cells and blood plasma.

17. The method for predicting a prognostic indications of pancreatic cancer patient according to claim 15,
wherein when the object is gene locus, the method of isolating the sample includes RNA Extraction, a DNA extraction, a polymerase chain reaction (PCR), and a reverse transcription polymerase chain reaction (RT-PCT);
wherein when the object is protein, the method of isolating the sample includes a process of cell lysis, centrifugation, precipitation, proteinase digestion, and isolation of subcellular fractions;
wherein when the object is blood cell, the method of isolating the sample includes a process of flow cytometry, hemocytometer, blood smear.

18. The method for predicting a prognostic indications of pancreatic cancer patient according to claim 15, wherein the clinical manifestation includes received surgery, systolic pressure, diastolic pressure, 9-item-report patient health questionnaire (PHQ-9).

19. The method for predicting a prognostic indications of pancreatic cancer patient according to claim 15, wherein the model includes Genetic Risk Score, Univariable Analysis, Multivariable Analysis, Dominant Model, Least Absolute Shrinkage and Selection Operator, Kaplan-Meier Curve, Cox Proportional Hazard Model, Akaike Information Criterion, and Bayesian Information Criterion.
